(19) United States
(12) Patent Application Publication
(10) Pub. No.: US 2009/0047371 A1
(43) Pub. Date: Feb. 19, 2009

(54) PHARMACEUTICAL COMPOSITION COMPRISING CURCUMIN AND RESVERATROL AND USES THEREOF IN MEDICAL FIELD

(75) Inventors: Damiano Turini, Firenze (IT); Stefan Coccoloni, Firenze (IT)

Correspondence Address: YOUNG & THOMPSON, 209 Madison Street, Suite 500, ALEXANDRIA, VA, 22314, US

(73) Assignee: SAFI INVESTMENT HOLDING AG

(21) Appl. No.: 12/067,291

(22) PCT Filed: Feb. 21, 2006
(86) PCT No.: PCT/IT06/00093

§ 371 Date: Mar. 19, 2008

(30) Foreign Application Priority Data
Feb. 21, 2005 (IT) FI2005A000031

(51) Int. Cl. A61K 31/12; A61K 31/355; A61K 31/375; A61K 31/165; A61K 36/73; A61K 36/70; A61P 17/06

(52) U.S. Cl. 424/765; 514/679; 514/458; 514/474; 514/627

(57) ABSTRACT

The present invention concerns a pharmaceutical composition containing curcumin and resveratrol and its application in the medical field. In particular, the composition according to the invention can be advantageously employed for preventing ageing and vascular diseases, for the treatment and the prophylaxis of cancers as prostate carcinoma, of skin diseases as psoriasis, and of the piliferous system as hair loss.

The present invention deals with a pharmaceutical composition comprising curcumin and resveratrol and uses thereof in medical field. More specifically, the composition object of the invention can be advantageously employed for retarding ageing, for the prevention of vascular diseases, for the treatment and prevention of cancers as, for example, prostate carcinoma, skin diseases as psoriasis, and diseases of the piliferous system as hair loss.

Resveratrol is a polyphenol present in grapes, especially in its peel and in its seeds, but also in a plant, the Polygonum Cuspidatum. Numbers of scientific researches have shown the in vitro efficacy of the Resveratrol in reducing the proliferation of human carcinogenic cells (Annals of the New York Academy of Science 957:210-229 (2002); Anticancer Res. 2004 September-October, 24(5A):2783-840; J. Urol. 2002 August 168(2):748-55; Biochem Pharmacol 2004 Sep. 15 68(6):1113-8; Drugs exp clin Res 2003 29(5-6):257-61). Moreover, the regular use of red wine in France could explain the so-called “French Paradox”, that is the low incidence of coronary diseases in French population even if its diet has a high fat content.

The researches carried out by D. Sinclair and others, published on the magazine NATURE ((2789-Sep. 7, 2004-VBCKNELL-111861), aroused great interest by considering the opportunity of a life extension in good conditions. The researches are based on data dated many years ago that showed that a controlled diet prolonged life of the Drosophila Melanogaster, even if as side effect the animal manifested drowsiness. It has been shown that the admixture of resveratrol to diet gives the same results of a controlled diet, but eliminating any negative side effect.

Moreover Sinclair, who discovered the ability of Resveratrol of prolonging the life of yeasts, tried it on fruit-flies and small worms that have biological processes similar to the human ones. He ascertained an increased activity and fertility, together with a greater egg production.

Curcumin is an extract of the root of the Curcumea Longa, a plant well known since a long time for its pharmaceutical properties. Nowadays curcumin is well known for its power of reducing cholesterol, its diuretic, choleretic, anti-inflammatory ability and for ameliorating the general conditions of patients treated with chemotherapy for neoplasia.

Particular feature of Curcumin is to belong to the capsaicinoids family, substances characterized by a pungent taste, similar to the one of pepper. From a point of view of molecular biology, the presence of a vanilloid receptor on the cell membrane and close to the mitochondrial structures can explain the induction of apoptosis or planned death of cells that would have become neoplastic. Moreover, in the Central Nervous System there are various receptors of capsaicinoids and this could explain the enhancement of the anti-aging action of resveratrol by curcumin.

The authors of the present invention, starting from the recognized anti-inflammable and anti-neoplastic action of resveratrol and curcumin, have tested two mixtures with an antioxidant action. The first one is made of basically resveratrol (called Resverage) and the other one made of resveratrol and curcumin (called Capsures). The mixtures have been packaged in capsules for oral use. At the beginning the compounds have been administered to patients with prostate carcinoma insensible to hormones. The choice of these patients depends of the fact that the evolution of neoplasia can easily be monitored by the prostate-specific antigens (PSA).

The group of patients that has assumed only resveratrol didn't show any modification on the PSA, while the group treated with resveratrol and curcumin showed with great surprise a reduction, sometimes also relevant, in variable time periods.

Moreover, during this testing, the patients treated with resveratrol and curcumin surprisingly showed positive improvements in the treatment of psoriasis and the attenuation and even a stop of the hair loss, a reduction of the hair graying and the rebirth of hair in bald areas sometimes even of the original color too. This aspect polarized the attention and the mechanism that ruled the association of the two substances became the object of the studies. The physic and pathologic basis of the action of the association of resveratrol and curcumin can be found in the relationship existing between the congenital hypo-sensitization of the sensory fibers of rats and the skin lesions similar to psoriatic lesions. In that case, the administration of a substance that reactivates the skin tropism would open new ways to the treatment of skin lesions. A possible theory is that it is possible to stimulate the peripheral sensory fibers with tropic function by associating curcumin and resveratrol. Regarding its action on hair growth, even if not yet known, it is probably linked to the release of P substance. The growth and the pigmentation of the hair follicle would depend on mesenchymal-epithelium-neiroderic interactions not very well-known yet, where the P substance would stimulate the hair growth in vivo (Ralf Paus et al Investigation Labs, Vol 71, No 1, p. 135, 1994).

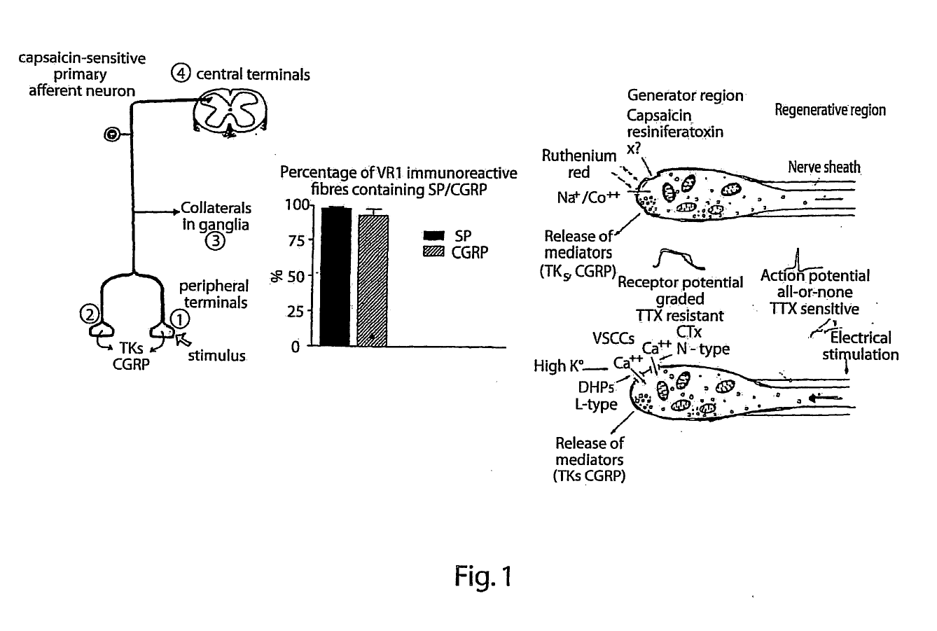

FIG. 1 shows a scheme of the capsaicin-sensible sensory neuron and the localization of tackykinins (P substance and Neurokinin A) and CGRP with the receptors TRPV 1. The scheme would show the double function, afferent and efferent, of the sensorial fibers with the release of neuropeptides from the terminal like the P substance, Neurokinin A and CGRP (Calcitonin gene related peptides). Researches are progressing about the use of high dosages of Curcumin for the treatment of colon cancer, but it seems like any effect on the hair growth has been shown; Effect that instead has been obtained thanks to the administration of curcumin together with resveratrol.

Therefore, the object of the present invention is a pharmaceutical composition comprising resveratrol and/or the root of polygonum cuspidatum, containing resveratrol, and curcumin and/or root of the curcuma longa, containing curcumin, as active principles, in association with one or more adjuvant and/or excipients pharmacologically acceptable. The root of powdered polygonum cuspidatum, a plant commonly used in the traditional Chinese and Japanese medicine as circulatory tonic, is particularly rich in resveratrol. Epidemiological and in vitro studies suggest that its use reduces the incidence of cardiovascular diseases and cancers. The powdered root of Curcuma longa, a plant diffused in the oriental medicine, is very rich in active elements as curcuminoids, the elements responsible for the characteristic yellow color and probably for the anti-inflammatory, antiviral and detoxicant effects too.

According to the invention, the pharmaceutical composition can comprise also vitamin E and/or wheat germs containing vitamin E. In fact, the powdered wheat germs are one of the richest natural resources of vitamin E (or tocopherol) and are one of the most effective antioxidants against free radicals, the aggressive substances that proliferate in our body because of the action of pollution, inaccurate diets, sun or just age.

Moreover, according to the invention, the pharmaceutical composition can also comprise vitamin C and/or berries of Rosa canina containing vitamin C. In fact the powdered berry of Rosa canina is a natural source of C vitamin, important for the good functioning of the immune system and an excellent antioxidant too. The vitamin C fights any kind of infection and facilitates the repair of the connective tissue. The modern living conditions, the high level of air pollution, smoking and passive smoking, increase our need of vitamin C.

According to another embodiment of the present invention, the pharmaceutical composition can also comprise Capsaicin and/or the fruit of capsicum annum, which contains capsaicin. Capsicum annum, its powdered fruit in particular, is rich in important nutritional substances as flavonoids, vitamins (C, E, PP, and K), lecithins, mineral salts and capsaicin, an alkaloid responsible for its particular taste and for its activating properties. The extract of capsicum seems to be very effective in stimulating the vitality of the tissues and in activating the venous and capillary circulation.

In particular, in the composition according to this invention, the content of resveratrol can range from 3 to 6 mg; of Curcumin from 15 to 30 mg; of vitamin E from 50 to 100 mg; of vitamin C from 10 to 30 mg; of capsaicin from 2 to 5 mg; of powdered wheat germs from 50 to 450 mg, preferably from 100 to 350 mg; of powdered berries of Rosa canina from 50 to 450 mg, preferably from 100 to 350 mg; of the powdered fruit of capsicum annum from 50 to 450, preferably from 100 to 350 mg; of the powdered root of curcuma longa from 1 to 100 mg, preferably from 50 to 100 mg; of the powdered root of polygonum cuspidatum from 1 to 100 mg, preferably from 50 to 100 mg.

The abovementioned quantities are the quantities contained in one dosage unit.

According to a preferred embodiment of the invention, the composition comprises Rosa canina (titrated dried extract) 10%; natural vitamin C 150.00 mg.; synthetic vitamin C 15.00 mg; Curcuma (titrated dried extract) 95%; Curcumin 20.60 mg; synthetic Curcumin 20.00 mg; Poligonum cuspidatum (titrated dried extract) 98%; resveratrol 5.10 mg; synthetic resveratrol 5.00 mg.; wheat germ oil 60.00 mg.; Capsicum (titrated dried extract) 60% in capsaicin 5.00 mg.; synthetic capsaicin 3.00 mg.

The composition according to the invention can be presented in capsules, pills, solution and emulsion. If in capsules or pills, the suggested dosage is one or two units, before meals.

Further object of the present invention is the use of the composition for the cancer treatment, in particular the treatment of prostate carcinoma. Moreover, the composition according to the invention can be advantageously employed for the treatment of skin diseases, in particular of psoriasis, for the treatment and the prevention from ageing, for the treatment and the prophylaxis of diseases of the piliferous system, in particular of alopecia.

Another object of the present invention is a combination comprising resveratrol and curcumin for the simultaneous, separate or sequential use in the treatment of cancer, as for example, the prostate carcinoma, in the treatment of skin diseases as psoriasis and for the prevention and the treatment of alopecia.

It is understood that the abovementioned examples are just an exemplification offered to illustrate the practical usages of the invention. The invention in fact can vary in its dosages and in its components, adding other components similar to the one suggested, but without altering the conceptual basis of the invention.

The present invention will now be described by way of information, not restrictively, in its preferred ways of presentation, with a particular reference to the attached pictures, where:

FIG. 1 shows a scheme of the capsaicinoids-sensible sensory neuron, and the localization of tackykinins (substance P and Neurokinin A) and CGRP with the receptor TRPV 1.

### EXAMPLE 1

### Study on the Effect of Resveratrol and the Association of Resveratrol and Curcumin on the Prostate-Specific Antigen (PSA) in Patients Affected by Prostate Carcinoma, on Hair Growth and on Psoriasis.

A composition has been prepared. It contains RESVERATROL (poligonum cuspidatum) and other antioxidants and it is called “Resverage”. Its ingredients are: Rosa canina (titrated dried extract) 10%; natural vitamin C 200.00 mg; synthetic vitamin C 20.00 mg; wheat germ oil 60.00 mg; poligonum cuspidatum (titrated dried extract) 98%; resveratrol 8.20 mg; synthetic resveratrol 8.00 mg. The administration capsules of this composition to 5 patients did not give any positive result. Thus, another composition containing resveratrol and curcumin (Curcuma longa) has been prepared and tested on a bigger group of patients.

In detail the composition was composed of:
Rosa canina (titrated dried extract) 10%, natural vitamin C 150.00 mg., synthetic vitamin C 15.00;
Curcuma (titrated dried extract) 95%, Curcumin 20.60; synthetic Curcumin 20.00 mg
Poligonum cuspidatum (titrated dried extract) 98%; resveratrol 5.10 mg.; Synthetic resveratrol 5.00 mg
Wheat germ oil 60.00 mg, capsicum (titrated dried extract) 60% in capsaicin 5.00 mg; synthetic capsaicin 3.00 mg.
Ingredients: dicalcic phosphate, fruits of rosa canina (titrated dried extract) maltodextrins 10%; natural vitamin C, wheat germ oil plv titrated 40% (triticum vulgaris modified amylum, microcrystalline cellulose, Curcuma rhizome (titrated dried extract) Curcuma Longa L. maltodextrins 95%; Curcumin, anti-agglomerative, vegetal stearate magnesium; coating agents: hydroxyl-propyl-methyl cellulose, glycerol, titanium dioxide; Capsicum fruit (titrated dried extract) (capsicum frutescens, capsicum annum) 60% in capsaicin, Poligonum Cuspidatum root (dried extract) 98%, resveratrol.

In this testing, apart from a relevant anti-cancer action on hormone-insensible prostate neoplasia, an effect on some patients has also been the stop of the hair loss and the growth of hair, sometimes of the original color too. Therefore patients without any neoplastic disease, but affected by alopecia or hair loss, have been included to the testing.

Materials and Methods

The group treated only with resveratrol didn't show any relevant variation, except from two cases that showed a slight decrease.

Group treated with resveratrol: 5 patient, aged between 64 and 76 years old, affected by a hormone-independent prostate carcinoma have been treated with Resverage, 4 pills a day, for a period that ranged from 30 and 90 days. The basic PSA varied from 25/220 mg/ml and it has been monitored for the 15 days following the treatment period. The patients tolerated the therapy without showing any nuisance. Table n°1 shows the results obtained with the treatment with Resverage.

TABLE 1

| Age | Reason for | | |
|---|---|---|---|
| Sex | the treatment | Dosage Duration | Results |
| 64 | PROSTATE | 4 CAPSULES | NO VARIATION |
| MAN | CARCINOMA | A DAY FOR | ON PSA VALUE |
| | IN SITU | 30 DAYS | |
| 70 | PROSTATE | 4 CAPSULES | NO VARIATION |
| MAN | CARCINOMA | A DAY FOR | ON PSA VALUE |
| | | 30 DAYS | |
| 72 | PROSTATE | 4 CAPSULES | NO VARIATION |
| MAN | CARCINOMA | A DAY FOR | ON PSA VALUE |
| | | 30 DAYS | |
| 75 | PROSTATE | | NO VARIATION |
| MAN | CARCINOMA | | ON PSA VALUE |
| 78 | PROSTATE | 4 CAPSULES | SLIGTH |
| MAN | CARCINOMA | A DAY FOR | VARIATION ON |
| | | 30 DAYS | PSA VALUE |

Group treated with curcumin e resveratrol: 20 patients, some of them affected by prostate neoplasia and/or by hair loss, treated with curcumin and resveratrol for a period ranging from 30 days to 6 months. The results are displayed on table n°2:

TABLE 2

| Age | Reason for | Dosage | |
|---|---|---|---|
| Sex | the treatment | Duration | Results |
| 67 | PROSTATE | 4 CAPSULES/DAY | REDUCTION OF PSA |
| MAN | CARCINOMA | 2 CYCLES OF 60 | VALUE. |
| | | DAYS | HAIR GROWTH IN BALD |
| | | | AREAS. |
| 69 | PROSTATE | 4 CAPSULES/DAY | REDUCTION OF PSA |
| MAN | CARCINOMA | FOR 6 MONTHS | VALUE. |
| | | | HAIR GROWTH OF THE |
| | | | ORIGINAL COLOUR IN |
| | | | BALD AREAS. |
| 46 | PROSTRATITIS | 2 CAPSULES/DAY | STOP OF HAIR LOSS, |
| MAN | | FOR 6 MONTHS | PSORIASIS |
| | | | DISAPPEARED. |
| 71 | PROSTATE | 2 CAPSULES/DAY | STOP OF HAIR LOSS, |
| MAN | CARCINOMA | FOR 3 MONTHS | PSORIASIS |
| | | | DISAPPEARED, |
| | | | REDUCTION OF PSA |
| | | | VALUE |
| 54 | HAIR LOSS | 2 CAPSULES/DAY | STOP OF HAIR LOSS |
| MAN | | FOR 30 DAYS | |
| 64 | PROSTRATITIS | 6 CAPSULES/DAY | STOP OF HAIR LOSS, |
| MAN | | FOR 30 DAYS | HAIR GROWTH, CLINIC |
| | | | CONDITIONS |
| | | | IMPROVEMENT, ANY |
| | | | VARIATION OF PSA |
| | | | VALUE |
| 56 | HAIR LOSS | 1 CAPSULE/DAY | STOP OF HAIR LOSS, |
| MAN | | FOR 4 MONTHS | HAIR GROWTH |
| 32 | HAIR LOSS | 2 CAPSULES/DAY | STOP OF HAIR LOSS, |
| MAN | | FOR 2 MONTHS | HAIR GROWTH |
| 75 | PROSTRATITIS | 2 CAPSULES/DAY | CLINIC CONDITIONS |
| MAN | | FOR 3 MONTHS | IMPROVEMENT, |
| | | | STOP OF HAIR LOSS, |
| | | | HAIR GROWTH |
| 66 | VOLOUNTEER | 2 CAPSULES/DAY | STOP OF HAIR LOSS, |
| MAN | | FOR 4 MONTHS | ORIGINAL COLOUR HAIR |
| | | | GROWTH |
| 69 | HAIR LOSS | 2 CAPSULES/DAY | HAIR GROWTH |
| WOMAN | | FOR 4 MONTHS | |
| 39 | HAIR LOSS | 2 CAPSULES/DAY | STOP OF HAIR LOSS, |
| WOMAN | | 2 CYCLES OF 45 | HAIR GROWTH |
| | | DAYS | |
| 29 | PROSTRATITIS | 4 CAPSULES/DAY | NOT VALUABLE |
| MAN | | INTERRUPTION | |
| | | OF THE | |
| | | TREATMENT FOR | |
| | | INTOLERANCE | |
| 28 | HAIR LOSS | 2 CAPSULES/DAY | NOT VALUABLE |
| MAN | | FOR 10 DAYS | |
| | | INTERRUPTION | |
| | | OF THE | |
| | | TREATMENT FOR | |
| | | INTOLERANCE | |
| 64 | HAIR LOSS | 4 CAPSULES/DAY | STOP OF HAIR LOSS, |
| WOMAN | | FOR 2 MONTHS | HAIR GROWTH |
| 75 | PROSTATE | 2 CAPSULES/DAY | REDUCTION OF PSA |
| MAN | CARCINOMA | FOR 2 MONTHS | VALUE, HAIR GROWTH |
| 75 | HAIR LOSS | 2 CAPSULES/DAY | STOP OF HAIR LOSS, |
| WOMAN | | FOR 3 MONTHS | HAIR GROWTH |
| 80 | PROSTATE | 2 CAPSULES/DAY | REDUCTION OF PSA |
| MAN | CARCINOMA AND | FOR 4 MONTHS | VALUE, |
| | INTOLERANCE TO | | ANY ACTION ON HAIR |
| | ANTI- | | |
| | ANDROGENICS | | |
| 65 | HAIR LOSS | 2 CAPSULES/DAY | STOP OF HAIR LOSS, |
| WOMAN | | FOR 3 MONTHS | HAIR GROWTH |

We comment the most startling cases of table 2:

Case 2: 69 years old man operated of radical prostatectomy for carcinoma 10 years before starting the treatment with curcumin and resveratrol (Capsures). Anatomic-clinic stage at the moment of the intervention: T3; Gleason 5+4. During the period between the operation and our testing, the patient had had various treatments (total androgenic block, anti-androgens radiant therapy). Before the treatment with Capsures, the PSA value was at 3300 mg/ml, without answer to the traditional treatments. After the administration for 30 days of Capsures, 4 pills a day, the value of PSA decreased to 1500 and then to 700 after 60 days. Afterwards, the patient interrupted the treatment and his PSA value increased until the patient restarted the treatment with Capsures again.

Case 4: 71 years old man operated for a prostate and vesicle carcinoma 10 years before, with a PSA value at 62 mg/ml. The administration of a slight dose of antiandrogen for 5 days and then of Capsures of 4 pills a day let to a reduction in the level of PSA after 60 days to 7.4 and then to 6.80. The patient suffered also of psoriasis of the scalp, that disappeared after the treatment and that by now, after 8 months has not reappeared.

Case 6: 64 years old man affected by prostratitis that tried various treatments. After the treatment with Capsures, 6 capsules a day, after 30 days had no more perineal pain and had a hair growth.

Case 14: 75 years old man, operated 4 years before for radical prostatectomy, intolerant to hormonal treatment he has been treated with Capsures, 4 pills a day for 60 days. His PSA value reduced from 4.5 to 2.4.

In one case, the Capsures treatment caused an increase of PSA. He was a patient with spread metastasis but his clinic conditions improved.

As far as its action on hair is concerned, some patients, especially women that were considerably loosing their hair, after 20 days of treatment confirmed to have had a reduction in their hair loss. In bald patients, the hair, sometimes of the original color restarted to grow even after the ablation of the white hair.

### CONSIDERATIONS AND CONCLUSIONS

The compound containing just resveratrol as active agent (Resverage) didn't cause a reduction of the PSA value and this can be due to the low bioavailability of the molecule. In the cases of prostate carcinoma the efficacy of the association of resveratrol with curcumin can be due to a higher concentration in the circulation and therefore in the tissues explained by the lipophilia of the active elements of curcumin.

The association of resveratrol and curcumin seems to have a synergic effect that enhances the anti-inflammatory power of the two active elements. It is an essential activity because in the prostate carcinoma there is a phlogistic action caused by hyperincretion of enzymes as the cyclooxygenase 1 and 2.

As far as the action on hair is concerned, it is unquestionably due to the administration of the two combined active elements, since in the compound with just resveratrol no result has been shown and in the many cases in India and in Europe where Curcumin has been employed, any effect on hair has never been reported. The most important aspect rarely highlighted is that curcuminoids are capsaicinoid substances.

It is demonstrated that hair has a sensory innervation defined capsaicin-sensitive, whose characteristic is that the C-amyelinated fibers have an afferent and efferent function (see scheme of picture n°1).

Those fibers, whose nerve cell is in the posterior roots, once stimulated release neuropeptides like the P substance, Neurokinin A and the Calcitonin Gene Related Peptide (CGRP). Experimental researches demonstrated that the desensibilization of those fibers leads to skin lesions localized in the cervical region of the rat. These experiments indicate the existence of a tropic function. It is probable that the loss of hair is a result of the deficit of neuropeptides released by the fibers that innervate the scalp.

Therefore it is possible that the administration of a substance that has as activity similar to capsaicin, as the Curcumin that metabolizes like an active curcumoid, can stimulate the sensorial fibers that have a tropic function, and that resveratrol has a vehicular function on the tissues. Therefore, the composition of the present invention is active on cases of hormone-insensible prostate carcinoma but in particular on hair, as our research has proven. The efficacy of the composition according to the present invention also depends on the great lyophilic properties of a capsaicinoid, curcumin on the anti-inflammatory effect of the two substances.

The interference of the hormonal action on hair loss is unquestionable, even if capsaicinoid-sensible sensory innervations are an ancestral phenomenon that does not depend on the hormonal mechanisms, but through a metabolic way different from the ordinary one. The growth and the pigmentation of the hair follicle seem to be controlled by epitelius-mesenchimoneuroechtodermmic interactions not very well known, but with an important role of the P substance on the hair growth in vivo.

1. Pharmaceutical composition comprising resveratrol and/or the root of polygonum cuspidatum containing resveratrol, and curcumin and/or root of the curcuma longa containing curcumin, as active principles in association with one or more pharmaceutically acceptable adjuvants and/or excipients.

2. Pharmaceutical composition according to claim 1, that further comprises vitamin E and/or wheat germs containing vitamin E.

3. Pharmaceutical composition according to claim 1, that further comprises vitamin C and/or berries of Rosa canina containing vitamin C.

4. Pharmaceutical composition according to claim 1, that further comprises capsaicin and/or the fruit of capsicum annum containing capsaicin.

5. Pharmaceutical composition according to claim 1, wherein the resveratrol content ranges from 3 to 6 mg.

6. Pharmaceutical composition according to claim 1, wherein the curcumin content ranges from 15 to 30 mg.

7. Pharmaceutical composition according to claim 1, wherein the content of vitamin E ranges from 50 to 100 mg.

8. Pharmaceutical composition according to claim 1, wherein the content of vitamin C ranges from 10 to 30 mg.

9. Pharmaceutical composition according to claim 1, wherein the content of capsaicin ranges from 2 to 5 mg.

10. Pharmaceutical composition according to claim 1, wherein the content of powdered wheat germs ranges from 50 to 450 mg.

11. Composition according to claim 10, wherein the content of powdered wheat germs ranges from 100 to 350 mg.

12. Composition according to claim 1, wherein the content of powdered berries of Rosa canina ranges from 50 to 450 mg.

13. Composition according to claim 12, wherein the content of powdered berries of Rosa canina ranges from 100 to 350 mg.

14. Composition according to claim 1, wherein the content of the powdered fruit of capsicum annum ranges from 50 to 450 mg.

15. Composition according to claim 14, wherein the content of powdered fruits of capsicum annum ranges from 100 to 350 mg.

16. Composition according to claim 1, wherein the content of powdered roots of Curcuma longa ranges from 1 to 100 mg.

17. Composition according to claim 16, wherein the content of powdered roots of Curcuma longa ranges from 50 to 100 mg.

18. Composition according to claim 1, wherein the content of powdered roots of polygonum cuspidatum varies from 1 to 100 mg.

19. Composition according to claim 18, wherein the content of powdered roots of ranges from 50 to 100 mg.

20. Pharmaceutical composition according to claim 1, comprising:
Rosa canina (titrated dried extracts) 10%; 150.00 mg of natural vitamin C; 15.00 mg of synthetic vitamin C,
Curcuma (titrated dried extract) 95%; 20.60 mg of Curcumin, 20.00 mg. of synthetic Curcumin,
Poligonum cuspidatum (titrated dried extract) 98%; 5.10 mg of resveratrol; 5.00 mg of synthetic resveratrol,
60.00 mg of wheat germ oil,
Capsicum, (titrated dry extract) 60% in Capsaicin 5 mg; 3.00 mg of synthetic capsaicin.

21. Composition according to claim 1 presented in the form of capsules, pills, solution or emulsion.

22-29. (canceled)

30. Combination comprising resveratrol and curcumin for the simultaneous, separate or sequential use in the treatment of cancer.

31. Combination according to claim 30, wherein cancer is the prostate cancer.

32. Combination comprising resveratrol and Curcumin for the simultaneous, separate or sequential use in the treatment of skin diseases.

33. Combination according to claim 32, wherein the skin disease is psoriasis.

34. Combination comprising resveratrol and curcumin for the simultaneous, separate or sequential use in the treatment and the prevention of alopecia.

35. A method for treating a patient having cancer, comprising administering to said patient in need thereof an effective amount of the pharmaceutical composition according to claim 1.

36. The method according to claim 35, wherein said patient has prostate carcinoma.

37. A method for treating a patient having a skin
disease, comprising administering to said patient in need thereof an effective amount of the pharmaceutical composition according to claim 1.

38. The method according to claim 37, wherein said patient has psoriasis.

39. A method for the treatment and prevention of ageing in a patient, comprising administering to said patient in need thereof an effective amount of the pharmaceutical composition according to claim 1.

40. A method for the treatment and prevention of vascular disease in a patient, comprising administering to said patient in need thereof an effective amount of the pharmaceutical composition according to claim 1.

41. A method for the treatment and prevention of a pilferous system disease in a patient, comprising administering to said patient in need thereof an effective amount of the pharmaceutical composition according to claim 1.

42. The method according to claim 41, wherein said patient has alopecia.
